(19) United States
(12) Patent Application Publication
(10) Pub. No.: US 2023/0324419 A1
(43) Pub. Date: Oct. 12, 2023

(54) METHOD FOR DIAGNOSING ACTING OF NERVONIC ACID AND USE THEREOF

(71) Applicant: BAO FENG BIOTECH (BEIJING) CO., LTD, Beijing (CN)

(72) Inventors: Xianyang CHEN, Beijing (CN); Wangting SONG, Beijing (CN); Jiarui HAN, Beijing (CN); Teng XUE, Beijing (CN)

(73) Assignee: BAO FENG BIOTECH (BEIJING) CO., LTD, Beijing (CN)

(21) Appl. No.: 18/021,229

(22) PCT Filed: Aug. 13, 2021
(86) PCT No.: PCT/CN2021/112421

§ 371 Date: Feb. 14, 2023

(30) Foreign Application Priority Data
Aug. 14, 2020 (CN) 202010816389.2

(51) Int. Cl. G01N 33/92; G01N 30/72

(52) U.S. Cl. CPC G01N 33/92; G01N 30/7233; G01N 2405/08; G01N 2800/285; G01N 2800/52

(57) ABSTRACT

This disclosure provides a method for diagnosing the action of nervonic acid and use thereof. The method for diagnosing acting of nervonic acid in acer truncatum bunge seed oil can be used to determine whether nervonic acid is absorbed and transformed by the body, and provides guidance for whether nervonic acid plays a role after intaking acer truncatum bunge seed oil.

### FIELD

The invention belongs to the field of biological detection technology, in particular to a method for diagnosing acting of nervonic acid and use thereof.

### BACKGROUND

Nervonic acid, is also known as selacholeic acid, with scientific name: cis-15-Tetracosenic acid, molecular formula: C₂₄H₄₆O₂, molecular weight: 366.6. Pure product of nervonic acid, which is a white needle shaped solid at room temperature, is an n-9 type long chain monoenoic fatty acid. Nervonic acid was first found in the nerve tissue of mammals, so it was named nervonic acid. Nervonic acid has a high content in nerve tissue and brain tissue, and is an important component of biofilm. It is usually used as a marker of medulla (white matter) in brain glycosides, and is involved in a variety of special physiological functions related to biofilm. Nervonic acid exists mainly in the form of sphingolipid and sphingomyelin in human brain protein, retina, sperm and nerve tissue. Due to the low efficiency of human body’s own synthesis of nervonic acid, nervonic acid can only be extracted from shark brain and a few plants in the nature, therefor it is an expensive raw material for medicine and health products in the world.

Studies in China and abroad have shown that nervonic acid is the core natural component of brain nerve cells and nerve tissue, and it is a special substance found in the world to promote the repair and regeneration of damaged nerve tissue. With the increase of age, the lack of nervonic acid in the body will increase the sequelae of stroke, senile dementia, cerebral palsy, brain atrophy, memory loss, insomnia and forgetfulness and other degenerative encephalopathy. The above diseases can be prevented by appropriate supplementation of nervonic acid in middle-aged and elderly people without disease. The human test results showed that, after taking nervonic acid once a day for one month, the test indexes of the experimental group including mind, picture, recognition, association, sence of touch, understanding were significantly improved compared with the control group, which indicated that nervonic acid could significantly improve the memory ability of the subjects. The antitumor activity of acer truncatum bunge seed oil with ascites tumor model showed that the average life extension rate of acer truncatum bunge seed oil on Ehrenbach’s ascites tumor mice is 81.19%. Therefore, the use of acer truncatum bunge seed oil can improve memory and cognitive ability.

The chemical essence of animal and plant oils is acylglycerol, and the most important one among them is triacylglycerol or triglyceride (TG). After taking acer truncatum bunge seed oil, whether the unique component it contains -nervonic acid can be absorbed and utilized by the body, at the same time, what metabolites TG-type nervonic acid will transform in the body, and what are the markers of acting, these very important questions have not been proved by specific studies so far, even there are few studies. Therefore, the present invention provides a method for determining the timing of acting of nervonic acid and at the same time searching for molecular markers that determine acting of nervonic acid.

### SUMMARY

In order to determine whether nervonic acid is effective in the organism, the present invention provides biomarkers for diagnosing acting of nervonic acid.

To achieve the above object, the invention adopts the following technical solution:

A Biomarker for diagnosing acting of nervonic acid, which include SM(d17:1/24:1) or/and Cer(d18:1/24:1(15Z)).

A use of the biomarker for diagnosing acting of nervonic acid described above in the preparation of reagents for detection of acting of nervonic acid.

According to the use described above, preferably, the content change of the biomarker for diagnosing acting of the nervonic acid in serum is more than 1.2 times before and after intaking acer truncatum bunge seed oil or products containing nervonic acid, indicating that acer truncatum bunge seed oil or products containing nervonic acid are effective.

According to the use described above, preferably, the content change of the biomarker SM(d17:1/24:1) for diagnosing acting of the nervonic acid in serum is more than 1.2 times before and after intaking acer truncatum bunge seed oil or products containing nervonic acid, indicating that acer truncatum bunge seed oil or products containing nervonic acid are effective.

According to the use described above, preferably, the content change of the biomarker Cer(d18:1/24:1(15Z) for diagnosing acting of the nervonic acid in serum is more than 1.3 times before and after intaking acer truncatum bunge seed oil or products containing nervonic acid, indicating that acer truncatum bunge seed oil or products containing nervonic acid are effective.

A detection method for diagnosing acting of nervonic acid, comprising detecting the content of the lipids SM(d17:1/24:1) and Cer(d18:1/24:1(15Z)) in serum samples isolated from subjects before and after intaking products containing nervonic acid.

According to the detection method described above, preferably, the content of lipid SM(d17:1/24:1) in serum after intaking products containing nervonic acid is 1.2 times or more than that before intaking products containing nervonic acid, the content of lipid Cer(d18:1/24:1(15Z)) in serum after intaking products containing nervonic acid is 1.3 times or more than that before intaking products containing nervonic acid, intaking products containing nervonic acid is determined to be effective.

According to the detection method described above, preferably, ultra-high performance liquid chromatography-mass spectrometry is used.

According to the detection method described above, preferably, the detection conditions of ultra-high performance liquid chromatography-mass spectrometry includes that C18 column is used, the mobile phase is 10 mM ammonium formate-0.1% formic acid-acetonitrile as phase A and 10 mM ammonium formate-0.1% formic acid-isopropanol-acetonitrile as phase B, the ion source temperature is 120° C., the desolubilization temperature is 600° C., the gas flow rate is 1000 L/h, and the flowing gas is nitrogen; the capillary voltage is 2.0 kV(+)/the cone voltage is 1.5 kV(-), and the cone voltage is 30 V.

A test kit for diagnosing acting of nervonic acid, which comprises SM(d17:1/24:1) and Cer(d18:1/24:1(15Z)) as standard substances, liquid A: containing 10 mM ammonium formate and 0.1% formic acid as solute, and acetonitrile and water with volume ratio of 60:40 as solvent; and liquid B: containing 10 mM ammonium formate and 0.1% formic acid as solute, and isopropanol and acetonitrile with volume ratio of 90:10.

A method for treating cognitive impairment in patients with demyelinating disease, which comprises detecting the content of SM(d17:1/24:1) A1 and the content of Cer(d18:1/24:1(15Z)) B1 in blood before supplementation with products containing nervonic acid, and the content of SM(d17:1/24:1) A2 and the content of Cer(d18:1/24:1(15Z)) B2 in blood after supplementation of nervonic acid products, if A2/A1≥1.2 and B2/B1≥1.3, the treatment of intaking products containing nervonic acid is effective; otherwise, the treatment is ineffective.

The present invention brings the following beneficial effects:

The present invention provides a biomarker for diagnosing acting of nervonic acid, and the detection of the biomarker can be used to determine whether nervonic acid is absorbed and transformed by the organism, providing guidance for whether intaking acer truncatum bunge seed oil or products containing nervonic acid is effective.

The invention also provides a use of a biomarker for the diagnosis of acting of nervonic acid in a detection reagent. It can be used to evaluate the effect of individual supplementation of nervonic acid products.

The invention further provides a detection and detection kit for diagnosing acting of nervonic acid, which can effectively evaluate the effect of individual supplementation of nervonic acid products, determine whether the supplemented nervonic acid products are effective, and then provide a basis for choosing to stop or continue intaking.

### DESCRIPTION OF DRAWINGS

FIG. 1 shows VIP > 1 compounds in positive and negative ion mode 1 day after acer truncatum bunge seed oil supplementation.

FIG. 2 shows the score plot of (O)PLS-DA in positive and negative ion mode 1 day after acer truncatum bunge seed oil supplementation.

FIG. 3 shows the S-plot in positive and negative ion mode 1 day after acer truncatum bunge seed oil supplementation.

FIG. 4 shows VIP > 1 samples in positive and negative ion mode 3 days after acer truncatum bunge seed oil supplementation.

FIG. 5 shows the score plot of (O)PLS-DA in positive and negative ion mode 3 days after aer truncatum bunge oil supplementation.

FIG. 6 shows the S-plot in positive and negative ion mode 3 days after aer truncatum bunge oil supplementation.

FIG. 7 shows VIP > 1 samples in positive and negative ion mode 7 days after acer truncatum bunge seed oil supplementation.

FIG. 8 shows the score plot of (O)PLS-DA in positive and negative ion mode 7 days after acer truncatum bunge seed oil supplementation.

FIG. 9 shows the S-plot in positive and negative ion mode 7 days after acer truncatum bunge seed oil supplementation.

FIG. 10 shows the changes of serum lipid SM(d17:1/24:1) containing nervonic acid chain different days after taking acer truncatum bunge seed oil.

FIG. 11 shows the changes of serum lipid Cer(d18:1/24:1(15Z)) containing nervonic acid chains different days after taking acer truncatum bunge seed oil.

### DETAILED DESCRIPTION

The following examples are used to further illustrate the invention, but shall not be construed as a limitation of the invention. Without deviating from the spirit and essence of the invention, any modification or substitution of the invention shall fall within the scope of the invention.

The acer truncatum bunge seed oil used in the invention was provided by BaoFeng Biotechnology (Beijing) Co. Ltd, and its nervonic acid content was detected by Pony, a third-party testing company, and the detection result showed cis-15-Tetracosenic acid contained was 6.89%. Unless otherwise specified, the technical means used in the examples are conventional means known to the person skilled in the field.

### Example 1

### I. Sample Collection

30 male SD rats aged 5-6 weeks were randomly divided into 2 groups, which were acer truncatum bunge seed oil group (NA group) and normal control group (CK group). After 1 week of adaptive feeding, rats in each group entered into the experiment. 0.03 g/kg/d of nervonic acid was administered to acer truncatum bunge seed oil group by gavage, once a day, according to the drug dose conversion method between human and rat. Blood was collected after 1 day of continuous administration. The normal control group was fed conventionally, and the sampling method was the same as above.

### II. Experimental Instruments and Reagents

1. Refrigerated centrifuge: Model D3024R, Scilogex Corporation, USA; 2. Vortex Oscillator: Model MX-S, Scilogex Corporation, USA; 3. High resolution Mass spectrometer: ESI-QTOF/MS; Model: Xevo G2-S Q-TOF; Manufacturer: Waters, Manchester, UK; 4. Ultra-high performance liquid chromatography: UPLC; Model: ACQUITY UPLC I-Class System; Manufacturer: Waters, Manchester, UK; 5. Data acquisition software: MassLynx4.1; Manufacturer: Waters; 6. Analysis and identification software: Progenesis QI; Manufacturer: Waters.

Experimental reagents: isopropanol, acetonitrile, formic acid, ammonia formate, leucine enkephalin, sodium formate; manufacturer: Fisher.

### III. Experimental Methods

### 1. Sample Pretreatment

The collected serum samples were thawed on ice, 200 µL plasma was extracted with 600 µL pre-cooled isopropanol, vortexed for 1 min, and incubated at room temperature for 10 min. Then the extraction mixture was stored overnight at -20° C., centrifuged at 4000r for 20 min, and the supernatant was transferred to a new centrifuge tube, diluted with isopropanol/acetonitrile/water (2:1:1, v:v:v) to 1:10. The samples were stored at -80° C. before LC-MS analysis. In addition, 10 µL of each extraction mixture was combined to prepare mixed plasma samples.

### 2. Ultra High Performance Liquid Chromatography-mass Spectrometry Method For Lipidomics

The mixed plasma samples were analyzed using ACQUITY UPLC(Waters, USA) connected to the Xevo-G2XS High Resolution Time of Flight (QTOF) Mass spectrometer (Waters) with ESI. CQUITY UPLC BEH C18 column (2.1× 100 mm, 1.7 µm, Waters), and mobile phase consisted of 10 mM ammonium formate-0.1% formic acid-acetonitrile (A, 60: 40, v/v) and 10 mM ammonium formate-0.1% formic acid-isopropanol-acetonitrile (B, 90:10, v/v) were used. Pilot trials with 10-, 15-, and 20-minute washout periods were conducted prior to the large-scale study to assess the potential impact of mobile phase composition and flow rate on lipid retention time. In PIM, rich lipid precursor ions and fragments separated in the same order, with similar peak shapes and ionic strengths. In addition, the mixed quality control (QC) with a 10-minute washout period also exhibited basal peak strength of precursor and fragment similar to the test sample. The flow rate of mobile phase was 0.4 mL/min. The column was initially eluted with 40% B, 43% B in 2 min according to linear gradient, and 50% B within 0.1 min, 54%B in 3.9 minutes according to linear gradient, and 70% B within 0.1 min. In the last part of the gradient, the amount of B increased to 99% in 1.9 minutes. Finally, solution B was returned to 40% within 0.1 min and the column was balanced for 1.9 min before the next sample injection. The lipids were detected by Xevo-G2XS QTOF mass spectrometer with a sample size of 5 µL/time, collection range of m/z50~1200 years, and collection time of 0.2 s/time. The ion source temperature was 120° C., the desolutizing temperature was 600° C., the gas flow rate was 1000 L/h, and nitrogen was used as the flowing gas. The capillary voltage was 2.0 kV(+)/the cone voltage was 1.5 kV(-), and the cone voltage was 30 V. A standard quality determination was carried out using leucine enkephalin and corrected with sodium formate solution. The samples were sorted randomly. A quality control (QC) sample was injected into every 10 samples and analyzed to investigate repeatability of the data.

### IV. Results

### 1. Multivariate Statistics Was Used to Search for Substances With Serum Differences

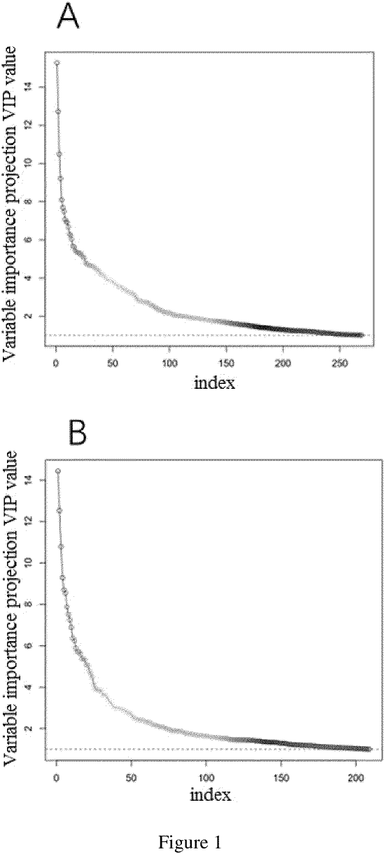

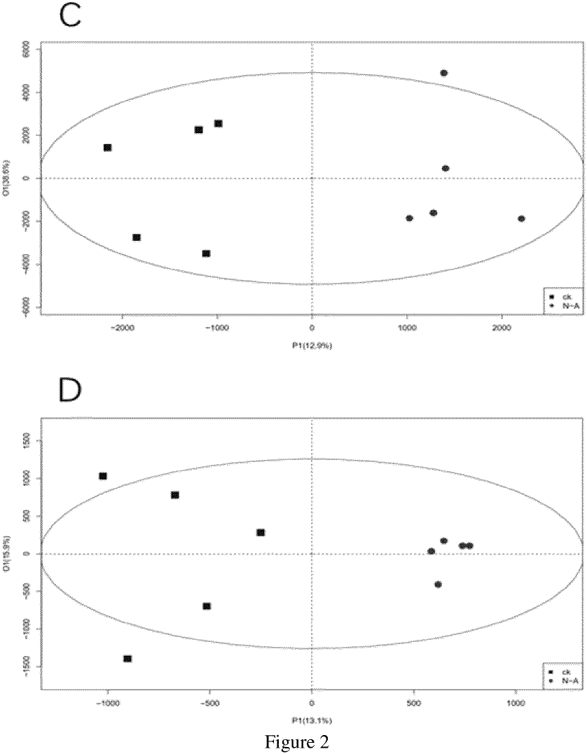

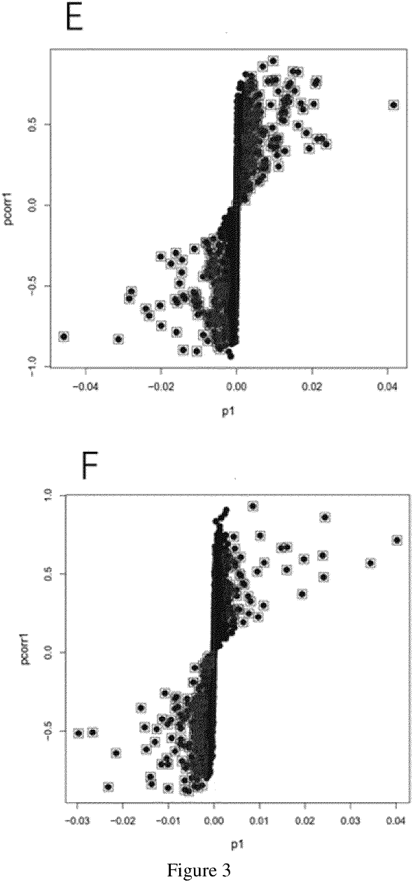

Orthogonal partial least squares discriminant analysis (OPLS-DA) combined with orthogonal signal correction (OSC) and partial least squares regression analysis (PLS-DA) methods were used to screen differential variables by removing uncorrelated differences. As shown in Figures, FIG. 1 shows the metabolite of VIP > 1 in positive and negative ion mode. Wherein, A was positive ion mode, B was negative ion mode, the VIP value was the variable importance projection of the first principal component of the orthogonal Partial least squares discriminant analysis (OPLS-DA), usually VIP > 1 was the commonly used evaluation criteria for metabolomics, as one of the criteria for screening differential metabolites; FIG. 2 shows the score plot of (O)PLS-DA in the positive and negative ion mode, and C was the score plot of (O)PLS-DA in the positive ion mode, (N-A represented the acer truncatum bunge seed oil group and CK represented the blank control group) and D was the score plot of (O)PLS-DA in the negative ion mode, that is, the score plot obtained by the first principal component and the second principal component in the two groups through the way of dimensionality reduction, the horizontal coordinate represented the difference between the groups, the vertical coordinate represented the difference within the group, and the two groups of results separation was good, indicating that this solution can be used. FIG. 3 shows the S-plot in positive and negative ion mode, E was the S-plot in positive ion mode, and F was the S-plot in negative ion mode. The horizontal coordinate represented the co-correlation coefficient between principal component and metabolite, and the vertical coordinate represented the correlation coefficient between principal component and metabolite, under the condition satisfying p<0.05 and VIP>1 at the same time, there were 24 differences in the negative ion mode and 49 differences in the positive ion mode.

In order to further narrow the scope, the VIP threshold was raised to 5, while reflecting that the difference in fold between normal and model was less than 0.7 times, or more than 1.4 times, and 5 compounds were finally obtained: TG(16:1(9Z)/22:5(7Z,10Z,13Z,16Z,19Z)/22:6(4Z,7Z,10Z,13Z,16Z,19Z)), TG(18:3(6Z,9Z,12Z)/20:3(8Z,11Z,14Z)/20:4,(5Z,8Z,11Z,14Z)), TG(14:1(9Z)/20:2(11Z,14Z)/22:5(7Z,10Z,13Z,16Z,19Z)), TG(16:0/20:3(8Z,11Z,14Z)/22:6(4Z,7Z,10Z,13Z,16Z,19Z)), TG(16:1(9Z)/18:4(6Z,9Z,12Z,15Z)/22:1(11Z)).

### 2. Jorden Index Analysis

Five compounds were calculated by youden Yoden index, AUC was used to reflect the diagnostic and predictive effect of a single indicator on the whole, so as to determine that these indicators were molecular markers. The results were shown in Table 1 below:

TABLE 1

Analysis of Yoden index of related lipids after 1 day of acer truncatum bunge seed oil supplementation

| No. | Name of compounds | AUC | specificity | sensitivity |
|---|---|---|---|---|
| R1 | TG(16:1(9Z)/22:5(7Z,10Z,13Z,16Z,19Z)/22:6(4Z,7Z,10Z,13Z,16 Z,19Z)) | 0.96 | 0.8 | 1 |
| R2 | TG(18:3(6Z,9Z,12Z)/20:3(8Z,11Z,14Z)/20:4(5Z,8Z,11Z,14Z)) | 0.96 | 0.8 | 1 |
| R3 | TG(14:1(9Z)/20:2(11Z,14Z)/22:5(7Z,10Z,13Z,16Z,19Z)) | 1 | 1 | 1 |
| R4 | TG(16:0/20:3(8Z,11Z,14Z)/22:6(4Z,7Z,10Z,13Z,16Z,19Z)) | 0.92 | 0.8 | 1 |
| R5 | TG(16:1(9Z)/18:4(6Z,9Z,12Z,15Z)/22:1(11Z)) | 0.88 | 0.8 | 1 |

Table 1 showed the area under the curve (AUC), sensitivity and specificity of a single metabolite for predicting a day after supplementation with the oil. Among the above five lipids, R3 showed the best predictive power (AUC=1).

The results showed that the above five compounds were specific molecular markers that appeared in blood one day after intaking acer truncatum bunge seed oil, and they did not contain compounds with 24:1 structure, indicating no effect.

### Example 2

Based on Example 1, the blood collected after continuous administration for 3 days was analyzed in this example, and the experimental operation and method were the same as in Example 1. The results were as follows:

### 1. Multivariate Statistics Were Used to Find Serum Differences

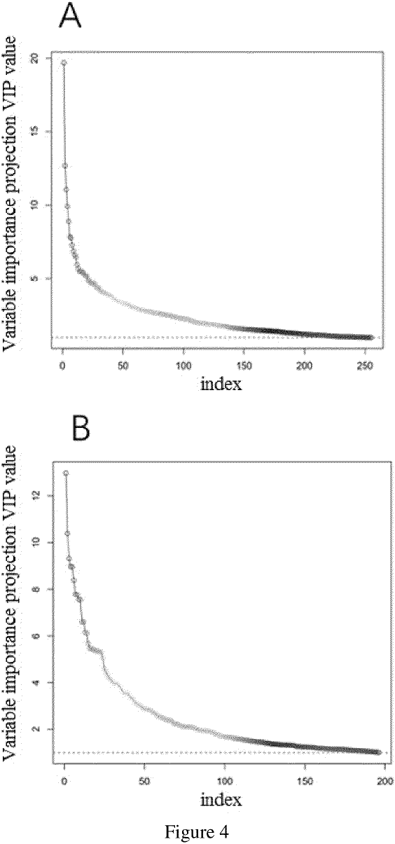

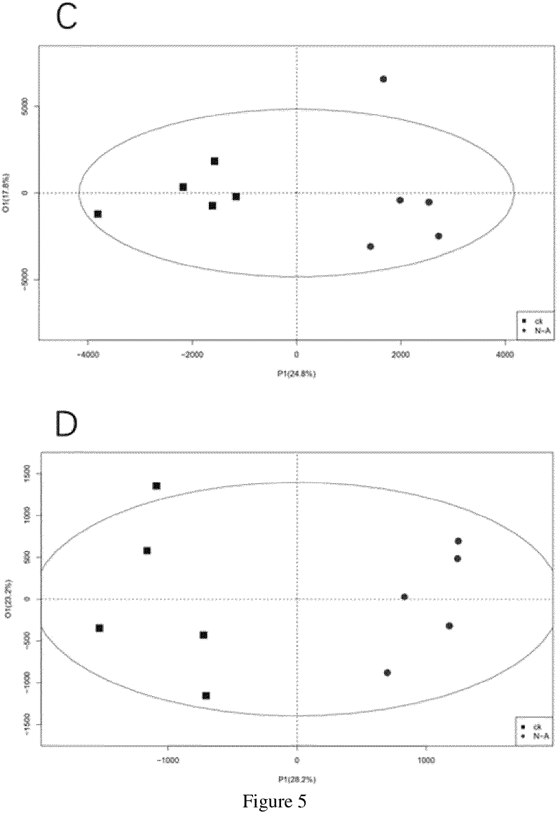

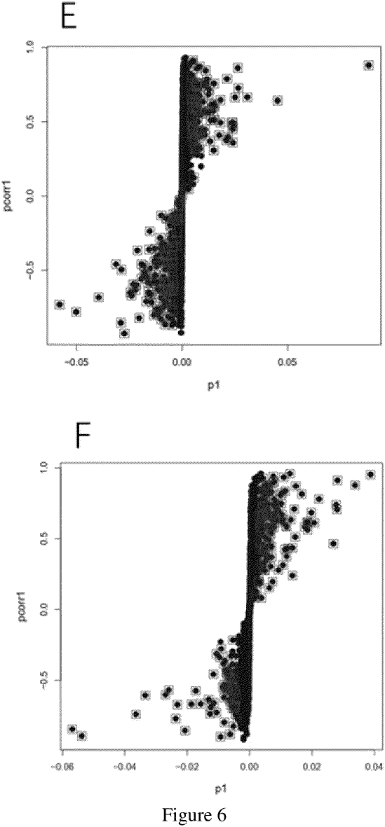

Orthogonal Partial least squares discriminant analysis (OPLS-DA) combined with orthogonal signal correction (OSC) and partial least squares regression analysis (PLS-DA) were used to screen differential variables by removing uncorrelated differences. The results are shown in FIG. 4, wherein A was VIP>1 metablite in positive ion mode, B was VIP>1 metablite in negative ion mode; The VIP value was the variable importance projection of the first principal component of the orthogonal partial least squares discriminant analysis (OPLS-DA), usually VIP>1 was the commonly used evaluation criteria for metabolomics, as one of the criteria for screening differential metabolites; FIG. 5 shows the score plot obtained by the first principal component and the second principal component in the two groups of acer truncatum bunge seed oil group (N-A) and blank control group (CK) through dimension reduction, where C was the score plot of (O)PLS-DA in positive ion mode, and D was the score plot of (O)PLS-DA in negative ion mode. The horizontal coordinate represented the difference between the groups, the vertical coordinate represented the difference within the group, and the two groups of results separation was good, indicating that this solution can be used. FIG. 6 was the S-plot, E was the S-polt in the positive ion mode, and F was the S-polt in the positive ion mode. The horizontal coordinate represented the correlation coefficient between principal component and metabolite, and the vertical coordinate represented the correlation coefficient between principal component and metabolite, under the condition satisfying p<0.05 and VIP>1 at the same time, there were 73 differences in the negative ion mode and 65 differences in the positive ion mode. In order to further narrow the scope, the VIP threshold was raised to 5, while reflecting the multiplier difference between normal and model was less than 0.7 times, or more than 1.4 times, and finally the following 9 compounds were obtained: SM(d17:1/24:1), 18:1-Glc-Campesterol; Cer(d18:1/24:1(15Z)); PI(18:0/20:3(8Z,11Z,14Z)); PS(21:0/20:2(11Z,14Z)); SM(d18:2/24:0); TG(17:2(9Z,12Z)/18:1(9Z)/22:1(11Z))[iso6]; PC(20:5(5Z,8Z,11Z,14Z,17Z)/20:3(8Z,11Z,14Z)); PC(20:3(5Z,8Z,11Z)/18:0).

### 2. Yoden Analysis

Then, for 9 compounds, the youden Yoden index was calculated to reflect the diagnostic and predictive effect of a single indicator on the whole, and the molecular markers were determined. The results were shown in Table 2

TABLE 2

Analysis of Yoden index of related lipids after 3 days of acer truncatum bunge seed oil supplementation

| No. | Name of compounds | AUC | specificity | sensitivity |
|---|---|---|---|---|
| R1 | SM(d17:1/24:1) | 1 | 1 | 1 |
| R2 | 18:1-Glc-Campesterol | 1 | 1 | 1 |
| R3 | Cer(d18:1/24:1(15Z)) | 1 | 1 | 1 |
| R4 | PI(18:0/20:3(8Z,11Z,14Z)) | 0.92 | 1 | 0.8 |
| R5 | PS(21:0/20:2(11Z,14Z)) | 0.88 | 1 | 0.8 |
| R6 | SM(d18:2/24:0) | 1 | 1 | 1 |
| R7 | TG(17:2(9Z,12Z)/18:1(9Z)/22:1(11Z)) | 1 | 1 | 1 |
| R8 | PC(20:5(5Z,8Z,11Z,14Z,17Z)/20:3(8Z,11Z,14Z)) | 0.96 | 0.8 | 1 |
| R9 | PC(20:3(5Z,8Z,11Z)/18:0) | 0.88 | 1 | 0.8 |

Table 2 showed the area under the curve (AUC), sensitivity and specificity of a single metabolite for predicting after 3-day supplementation with the oil. Relevant parameters showed that R1, R2, R3, R6 and R7 had the best predictive power among the above 9 lipids (AUC=1), indicating that they were molecular markers in blood. R1 and R3 contained 24:1, showing the presence of nervonic acid chains, and indicating that the oil began to take effect.

### Example 3

Based on Example 1, the blood collected 7 days after continuous administration was analyzed in this example. The experimental operation and method were the same as that of Example 1. The results were as follows:

### 1. Multivariate Statistics Were Used to Find Serum Differences

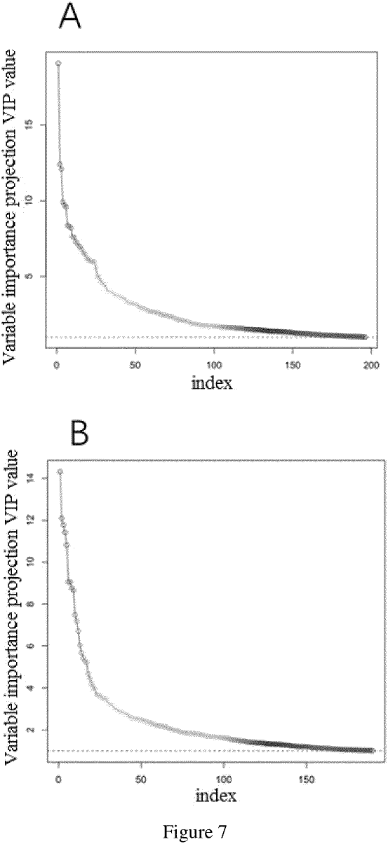

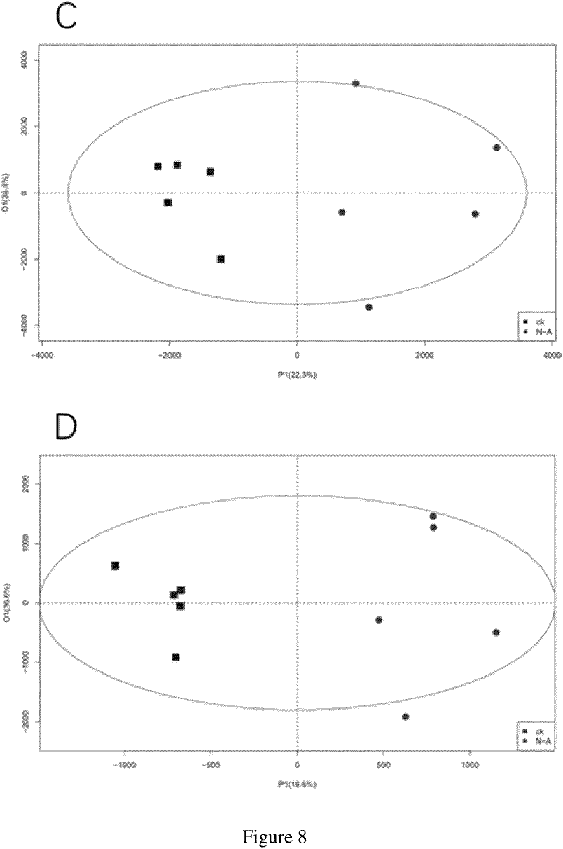

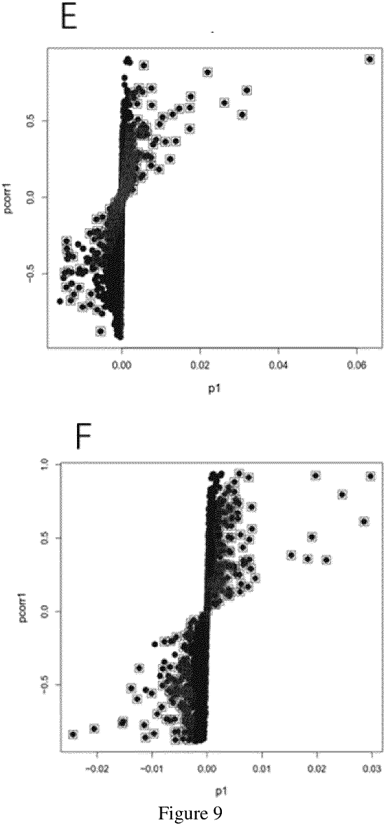

Orthogonal Partial least squares discriminant analysis (OPLS-DA) combined with orthogonal signal correction (OSC) and PLS-DA methods were used to screen differential variables by removing uncorrelated differences. FIG. 7 shows the metabolite of VIP > 1 in the positive and negative ion mode, wherein A was positive ion mode, B was negative ion sample, VIP value was the variable importance projection of the first principal component of the orthogonal partial least squares discriminant analysis (OPLS-DA), usually VIP>1 was the commonly used evaluation criteria for metabolomics, as one of the criteria for screening differential metabolites; FIG. 8 was the score plot of (O)PLS-DA in positive and negative ion mode, in which C was the score plot of (O)PLS-DA in positive ion mode, (N-A represented acer truncatum bunge seed oil group, CK represented blank control group) and D was the score plot of (O)PLS-DA in negative ion mode, that is, the score plot obtained by the first principal component and the second principal component in the two groups of the acer truncatum bunge seed oil group (N-A) and the blank control group (CK) through dimension reduction. The horizontal coordinate represented the difference between the groups, and the vertical coordinate represented the difference within the group, and the results of the two groups were well separated, indicating that this solution can be used. FIG. 9 shows the S-plot in the positive and negative ion mode, wherein E was the S-plot in the positive ion mode, and F was the S-plot in the negative ion mode. The x-coordinate represented the co-correlation coefficient between principal component and metabolite, and the y-coordinate represented the correlation coefficient between principal component and metabolite, under the condition satisfying p<0.05 and VIP>1 at the same time, there were 51 differences in the negative ion mode and 21 differences in the positive ion mode. To further narrow the range, the VIP threshold was raised to 5, while reflecting that the difference in fold between normal and model was less than 0.7 times, or more than 1.4 times, and finally the following 4 compounds were obtained: SM(d17:1/24:1), 1,2-didocosanoyl-sn-glycero-3-phosphosulfocholine, Cer(d18:1/24:1(15Z)), SM(d18:2/24:0).

### 2. Yoden Analysis

Then, the youden Yoden index was calculated for these four compounds to reflect the diagnostic and predictive effect of a single indicator on the whole. The results were shown in Table 3.

TABLE 3

Analysis of Yoden index of related lipids after 7 days of acer truncatum bunge seed oil supplementation

| No. | Name of compounds | AUC | specificity | sensitivity |
|---|---|---|---|---|
| R1 | SM(d17:1/24:1) | 1 | 1 | 1 |
| R2 | 1,2-didocosanoyl-sn-glycero-3-phosphosulfocholine | 1 | 1 | 1 |
| R3 | Cer(d18:1/24:1(15Z)) | 1 | 1 | 1 |
| R4 | SM(d18:2/24:0) | 1 | 1 | 1 |

Table 3 showed the area under the curve (AUC), sensitivity and specificity of a single metabolite for predicting 7 days after supplementation with the oil. The relevant parameters showed that among the four lipids, SM(d17:1/24:1), 1,2-didocosanoyl-sn-glycero-3-phosphosulfocholine, Cer(d18:1/24:1(15Z)) and SM(d18:2/24:0) exhibited the best predictive power (AUC=1), indicating that they were both biomarkers found in blood. 24:1 was found in SM(d17:1/24:1) and Cer(d18:1/24:1(15Z)), showing the presence of nervonic acid chains, and indicating the oil began to take effect.

Example 4 Changes of serum lipids containing nervonic acid chains after taking acer truncatum bunge seed oil for different days

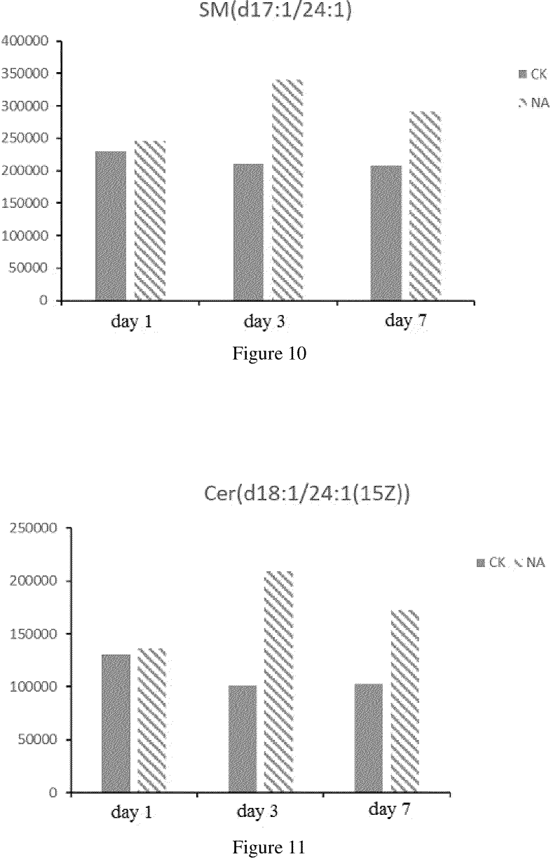

50 male SD rats aged 5-6 weeks were randomly divided into 2 groups, which were acer truncatum bunge seed oil group (NA group) and normal control group (CK group). After 1 week of adaptive feeding, rats in each group entered into the experiment. Acer truncatum bunge seed oil group was administrated by gavage according to the content of nervonic acid 0.03 g/kg/d, once a day, and blood was collected after 1, 3 and 7 days. The normal control group was fed routinely, and the blood was collected at the same time as the acer truncatum bunge seed oil group for the detection of lipid SM(d17:1/24:1) and Cer(d18:1/24:1(15Z)) with the nervonic acid chain. The detection results were shown in Table 4, wherein, the changes of SM(d17:1/24:1) were shown in FIG. 10. The test results of Cer(d18:1/24:1(15Z)) were shown in FIG. 11.

TABLE 4

| Change of SM(d17:1/24:1) and Cer(d18:1/24:1(15Z)) | Day 1 (NA1) | Day 3 (NA3) | Day 7 (NA7) | NA7/NA1 |
|---|---|---|---|---|
| SM(d17:1/24:1) | 245574.2 | 340912.8 | 291861.8 | 1.2 |
| Cer(d18:1/24:1(15Z)) | 135883.1 | 209352.5 | 172840.3 | 1.3 |

The results showed that the content of SM(d17:1/24:1) and Cer(d18:1/24:1(15Z)) lipids containing nervonic acid chains increased compared with the normal group. At the same time, there was no significant difference in the content of the two compounds between the third and the seventh day of the administration. Therefore, 3 days after intaking acer truncatum bunge seed oil, the effect was stable.

The results showed that compared with before taking acer truncatum bunge seed oil, the blood levels of SM(d17:1/24:1) or Cer(d18:1/24:1(15Z)) after taking acer truncatum bunge seed oil were more than 1.2 times or 1.3 times, proving that after taking acer truncatum bunge seed oil, the TG-type nervonic acid was absorbed by the body and converted into SM(d17:1/24:1) and Cer(d18:1/24:1(15Z)).

The above was a statistical proof that the TG-type nervonic acid contained in the oil was absorbed by the body and converted into SM(d17:1/24:1) and Cer(d18:1/24:1(15Z)). However, in specific cases, SM(d17:1/24:1) content of 7 rats did not change significantly on the first day of feeding of acer truncatum bunge seed oil, and Cer(d18:1/24:1(15Z)) content of 5 rats did not change significantly on the first day of feeding acer truncatum bunge seed oil. It was difficult to determine TG-type nervonic acid absorption by the body only from the change of one substance. However, when the change of SM(d17:1/24:1) content and Cer(d18:1/24:1(15Z)) content were combined in individuals, it was clear that TG type nervonic acid was absorbed by the body, indicating that, when SM(d17:1/24:1) was combined with Cer(d18:1/24:1(15Z)), the acting of nervonic acid can be determined more effectively and earlier.

### Example 5

A test kit for diagnosing acting of nervonic acid onset, included standard SM(d17:1/24:1) and Cer(d18:1/24:1(15Z)), 10 mM ammonium formate-0.1% formic acid-acetonitrile (A, acetonitrile: water=60:40, v/v), 10 mM ammonium formate-0.1% formic acid-isopropanol-acetonitrile (B, isopropanol: acetonitrile=90:10, v/v). wherein, 10 mM ammonium formate-0.1% formic acid-acetonitrile (A, acetonitrile: water=60:40, v/v) was prepared by dissolving 0.63 g ammonium formate and 10 g formic acid with acetonitrile-water solution (acetonitrile: water=60:40, v/v) to make total volume of 1000 mL.

10 mM ammonium formate-0.1% formic acid-isopropanol-acetonitrile (B, isopropanol: acetonitrile=90:10, v/v) was prepared by dissolving 0.63 g ammonium formate, 10 g formic acid with isopropanol-acetonitrile solution (isopropanol: acetonitrile=90:10, v/v) to make total volume of 1000 mL.

For sample detection, sample pretreatment and ultra-high performance liquid chromatography-mass spectrometry in Example 1 were used. At the same time, standard SM(d17:1/24:1) and Cer(d18:1/24:1(15Z)) were used as reference for detection.

### Example 6

For patients with demyelinating disease before and after supplementing with acer truncatum bunge seed oil or nervonic acid, and those who did not take acer truncatum bunge seed oil were set as control group, it was found that after 1 day of taking acer truncatum bunge seed oil, the content of serum lipid SM(d17:1/24:1) was 1.15 times that of SM(d17:1/24:1) before taking acer truncatum bunge seed oil, while the content of serum lipid Cer(d18:1/24:1(15Z)) was 1.23 times that of Cer(d18:1/24:1(15Z)) before taking acer truncatum bunge seed oil in individual patients, indicating that the single indicator could not be used to determine the effect of supplementing acer truncatum bunge seed oil or nervonic acid. It was found that after 7-day of taking acer truncatum bunge seed oil, the serum lipid SM(d17:1/24:1) content was 1.22 times that of SM(d17:1/24:1) before taking acer truncatum bunge seed oil, and the serum lipid Cer(d18:1/24:1(15Z) content was 1.45 times that of Cer(d18:1/24:1(15Z) before taking acer truncatum bunge seed oil. In contrast, the serum lipid SM(d17:1/24:1) content and Cer(d18:1/24:1(15Z) content in the control group decreased or basically did not change, indicating that intaking products containing nervonic acid can delay the speed of myelin sheath loss and repair the damaged nerve fibers, so as to improve cognitive function and memory.

What is claimed is:

1. A detection method for diagnosing acting of nervonic acid, comprising detecting the content of the lipids SM(d17:1/24:1) and Cer(d18:1/24:1(15Z)) in serum samples isolated from subjects before and after intaking products containing nervonic acid.

2. The detection method according to claim 1, wherein, the content of lipid SM(d17:1/24:1) in serum after intaking products containing nervonic acid is 1.2 times or more than that before intaking products containing nervonic acid, and the content of lipid Cer(d18:1/24:1(15Z)) in serum after intaking products containing nervonic acid is 1.3 times or more than that before intaking products containing nervonic acid, intaking products containing nervonic acid is determined to be effective.

3. The detection method according to claim 1, using ultra-high performance liquid chromatography-mass spectrometry.

4. The detection method according to claim 3, wherein, the detection conditions of ultra-high performance liquid chromatography-mass spectrometry includes that C18 column is used, the mobile phase is 10 mM ammonium formate-0.1% formic acid-acetonitrile as phase A and 10 mM ammonium formate-0.1% formic acid-isopropanol-acetonitrile as phase B, the ion source temperature is 120° C., the desolubilization temperature is 600° C., the gas flow rate is 1000 L/h, and the flowing gas is nitrogen; the capillary voltage is 2.0kV(+)/the cone voltage is 1.5 kV(-), and the cone voltage is 30 V.

5. A test kit for diagnosing acting of nervonic acid, comprising SM(d17:1/24:1) and Cer(d18:1/24:1(15Z)) as standard substances, liquid A: containing 10 mM ammonium formate and 0.1% formic acid as solute, and acetonitrile and water with volume ratio of 60:40 as solvent; and liquid B: containing 10 mM ammonium formate and 0.1% formic acid as solute, and isopropanol and acetonitrile with volume ratio of 90:10.

6. A method for treating cognitive impairment in patients with demyelinating disease, comprising detecting the content of SM(d17:1/24:1) A1 and the content of Cer(d18:1/24:1(15Z)) B1 in blood before supplementation with products containing nervonic acid, and the content of SM(d17:1/24:1) A2 and the content of Cer(d18:1/24:1(15Z)) B2 in blood after supplementation of nervonic acid products, if A2/A1≥1.2 and B2/B1≥1.3, the treatment of intaking products containing nervonic acid is effective; otherwise, the treatment is ineffective.
